(19) United States
(12) Patent Application Publication
(10) Pub. No.: US 2022/0022819 A1
(43) Pub. Date: Jan. 27, 2022

(54) BIOLOGICAL INFORMATION PROCESSING APPARATUS, METHOD, AND COMPUTER READABLE RECORDING MEDIUM

(71) Applicant: NEC Corporation, Minato-ku, Tokyo (JP)

(72) Inventors: Yuji OHNO, Tokyo (JP); Masahiro KUBO, Tokyo (JP); Toshinori HOSOI, Tokyo (JP)

(73) Assignee: NEC Corporation, Minato-ku, Tokyo (JP)

(21) Appl. No.: 17/428,102

(22) PCT Filed: Feb. 8, 2019
(86) PCT No.: PCT/JP2019/004660

§ 371 Date: Aug. 3, 2021

(51) Int. Cl. A61B 5/00; G16H 40/63

(52) U.S. Cl. CPC A61B 5/7282; G16H 40/63

(57) ABSTRACT

An internal state identification unit acquires sensor data of a person to be monitored, such as a patient, from a sensor group including one or a plurality of sensors. An identification model is a model for identifying an internal state of the person to be monitored. The identification model is generated using sensor data acquired in the past. The internal state identification unit identifies the internal state of the person to be monitored based on the acquired sensor data and the identification model. A determination unit determines whether or not a condition for generating another identification model is satisfied. A model generation unit generates, when the determination unit determines that the condition is satisfied, an identification model different from the identification model by using the sensor data of the person to be monitored acquired from the sensor group.

### TECHNICAL FIELD

The present disclosure relates to a biological information processing apparatus, a method, and a computer readable recording medium, and more particularly, to a biological information processing apparatus, a method, and a computer readable recording medium that perform processing on biological information acquired from a patient or the like.

### BACKGROUND ART

Patients hospitalized include, for example, those regarding which there is risk of their displaying problematic behavior, such as falling from a bed, removing an intubation tube, making strange noises, or committing acts of violence. Patients who display problematic behavior are often in a state called “a restless state” or “delirium”. Some medical workers such as nurses and care workers spend from 20 to 30% of their time dealing with hospitalized patients regarding which there is risk of their displaying problematic behavior. Consequently, their time for focusing on their primary care duties is reduced.

Patent Literature 1 discloses a biological information monitoring system that monitors biological information of a subject on a bed. The biological information monitoring system disclosed in Patent Literature 1 includes a physical condition judging unit. The physical condition judging unit judges the physical condition of the subject by using various types of biological information such as a body weight, a body motion, respiration, and a heart rate. The physical condition judging unit judges whether or not the subject is in a sleep state by applying various types of biological information of the subject to a function (model) representing whether or not the subject is in a sleep state, which has been trained using, for example, labeled training data. Alternatively, the physical condition judging unit judges whether the subject is in a delirium state based on body motion information and/or a respiratory rate of the subject.

### CITATION LIST

### Patent Literature

Patent Literature 1: Japanese Patent No. 6339711

### SUMMARY OF INVENTION

### Technical Problem

In Patent Literature 1, for example, a function representing sleep or wakefulness is prepared using data of a large number of pieces of biological information (labeled training data). However, Patent Literature 1 fails to disclose a modification of the trained function. For example, in a certain hospital, the relation between data of biological information and sleep or wakefulness may change when, for example, the ratio of the composition of the medical departments of the hospitalized patient changes. Further, the relation between data of biological information and sleep or wakefulness may change in accordance with seasonal changes. In such a case, when the prepared function is continuously used, a problem occurs in which the accuracy of a result of a determination of a physical condition is decreased.

The present disclosure has been made in view of the above-described circumstances and an object thereof is to provide a biological information processing apparatus, a method, and a computer readable recording medium that are capable of preventing or reducing a decrease in accuracy of a result of a determination of a physical condition.

### Solution to Problem

In order to achieve the aforementioned object, the present disclosure provides a biological information processing apparatus including: internal state identification means for acquiring sensor data of a person to be monitored from a sensor group including one or a plurality of sensors and identifying an internal state of the person to be monitored based on the acquired sensor data and an identification model for identifying the internal state of the person to be monitored, the identification model being generated using sensor data acquired in the past; determination means for determining whether or not a condition for generating another identification model different from an existing identification model is satisfied; and model generation means for generating, when the determination means determines that the condition is satisfied, an identification model by using the sensor data of the person to be monitored that is acquired from the sensor group, the identification model being different from the identification model used by the internal state identification means.

Further, the present disclosure provides a biological information processing method including: acquiring sensor data of a person to be monitored from a sensor group including one or a plurality of sensors and identifying an internal state of the person to be monitored based on the acquired sensor data and an identification model for identifying the internal state of the person to be monitored, the identification model being generated using sensor data acquired in the past; determining whether or not a condition for generating another identification model different from an existing identification model is satisfied; and generating, when it is determined that the condition is satisfied, an identification model by using the sensor data of the person to be monitored that is acquired from the sensor group, the identification model being different from the identification model used to identify the internal state.

The present disclosure provides a computer readable recording medium storing a program for causing a computer to: acquire sensor data of a person to be monitored from a sensor group including one or a plurality of sensors and identify an internal state of the person to be monitored based on the acquired sensor data and an identification model for identifying the internal state of the person to be monitored, the identification model being generated using sensor data acquired in the past; determine whether or not a condition for generating another identification model different from an existing identification model is satisfied; and generate, when it is determined that the condition is satisfied, an identification model by using the sensor data of the person to be monitored that is acquired from the sensor group, the identification model being different from the identification model used to identify the internal state.

### Advantageous Effects of Invention

The biological information processing apparatus, a method, and a computer readable recording medium according to the present disclosure are capable of preventing or reducing a decrease in accuracy of a result of a determination of a physical condition.

### BRIEF DESCRIPTION OF DRAWINGS

FIG. 1 is a block diagram schematically showing a biological information processing apparatus according to the present disclosure;

FIG. 2 is a block diagram showing a system including a biological information processing apparatus according to a first example embodiment of the present disclosure;

FIG. 3 is a graph showing a specific example of a restlessness score;

FIG. 4 is a flowchart showing an operation procedure in the first example embodiment;

FIG. 5 is a flowchart showing an operation procedure in a second example embodiment; and

FIG. 6 is a block diagram showing a configuration example of an information processing apparatus that can be used for the biological information processing apparatus.

### DESCRIPTION OF EMBODIMENTS

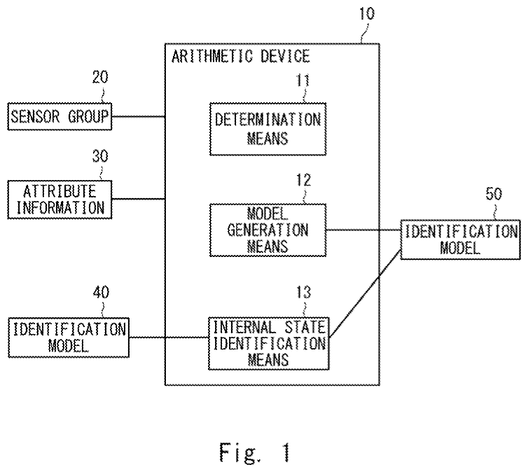

Prior to describing example embodiments according to the present disclosure, an overview of the example embodiments will be given. FIG. 1 schematically shows a biological information processing apparatus according to the present disclosure. A biological information processing apparatus 10 includes determination means 11, model generation means 12, and internal state identification means 13.

A sensor group 20 includes one or a plurality of sensors. The internal state identification means 13 acquires sensor data of a person to be monitored, such as a patient, from the sensor group 20. The internal state identification means 13 identifies the internal state of the person to be monitored based on the acquired sensor data and an identification model 40. It should be noted that the internal state of the person to be monitored refers to, for example, a state of the person to be monitored that cannot be directly determined by another person from the external state of the person to be monitored, and includes, for example, a mental state. Further, the identification model 40 is a model for identifying the internal state of the person to be monitored, which model is generated by using sensor data acquired in the past. Note that the sensor data acquired in the past means data acquired before the internal state of the person to be monitored is identified. The data acquired in the past includes data of the person to be monitored himself/herself, for example, data acquired when the person to be monitored himself/herself was in a facility or the like in the past. Alternatively, the data acquired in the past may be data acquired from a person different from the person to be monitored himself/herself, which data does not include the data of the person to be monitored himself/herself.

The determination means 11 determines whether or not a condition for generating another identification model different from an existing identification model is satisfied. When the determination means 11 determines that the condition for generating another identification model is satisfied, the model generation means 12 generates an identification model 50 different from the identification model 40 used by the internal state identification means 13 by using the sensor data of the person to be monitored that is acquired from the sensor group 20.

In the present disclosure, when the condition for generating another identification model is satisfied, the model generation means 12 generates the identification model 50 by using the sensor data of the person to be monitored that is acquired from the sensor group 20. The internal state identification means 13 can identify the internal state by using the generated identification model 50. Since the identification model 50 is generated using the sensor data acquired from the person to be monitored, the accuracy of a result of the identification performed when the identification model 50 is used may be higher than that of a result of the identification performed when the identification model 40 is used. In the present disclosure, since the identification model 50 is generated when the above condition is satisfied, it is possible to prevent or reduce a decrease in accuracy of a result of the identification of the internal state of the person to be monitored under the condition where the above condition is satisfied.

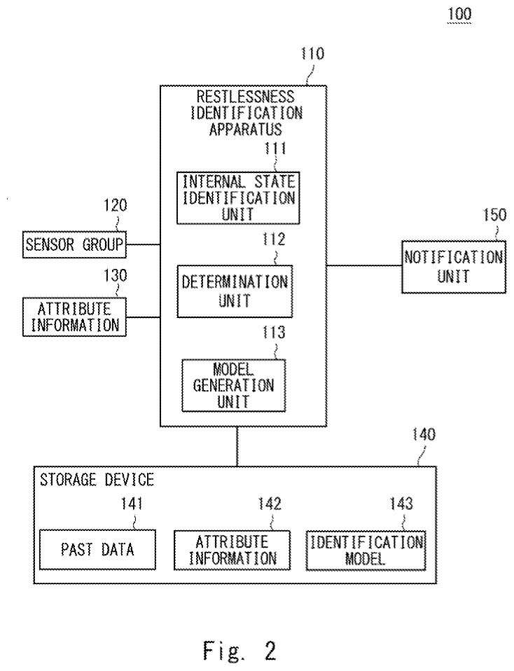

Hereinafter, example embodiments according to the present disclosure will be described in detail with reference to the drawings. FIG. 2 shows a biological information processing apparatus system including a biological information processing apparatus according to a first example embodiment of the present disclosure. A biological information processing system 100 includes a biological information processing apparatus (a restlessness identification apparatus) 110, a sensor group 120, a storage device 140, and a notification unit 150. The restlessness identification apparatus 110 is configured as a computer apparatus including, for example, a memory and a processor. The storage device 140 is configured as a storage device such as a Hard Disk Drive (HDD) or a Solid State Drive (SSD). The restlessness identification apparatus 110 corresponds to the biological information processing apparatus 10 shown in FIG. 1.

It should be noted that, according to the observation made by the present inventors, it has been found that, at least in the case of a neurosurgical patient, the patient is often in a state of restlessness (a restless state) in which he/she acts in an excessive manner before he/she actually displays problematic behavior. The “restless state” may include not only the state in which he/she acts in an excessive manner and is restless, but also a state in which he/she is not calm and a state in which it is not possible to control the patient's mind so that it is normal. As the restless state is caused by at least one of physical distress and delirium, it is assumed that the term “restless state” as used herein includes delirium.

Possible specific behavior of the patient in the restless state may include continuous movement of his/her hands and feet, trembling of his/her body, an unnatural focusing on some movement, saying something incoherent and inarticulate, and not listening to nurses or care workers. The restless state may also include behavior that is not harmful to the patient, for example, behavior associated with the patient's desire to urinate. In this example embodiment, the restlessness identification apparatus 110 identifies an internal state, including a mental state, of a person to be monitored, such as a patient. For example, the restlessness identification apparatus 110 identifies the restless state of the person to be monitored.

The storage device 140 stores past data 141, attribute information 142, and an identification model 143. The identification model 143 is an identification model (an identification parameter) for generating information indicating levels of the restless state from sensor data obtained from the sensor group 120. The levels of the restless state include, for example, a restless state, a normal state, and an unknown state that is used when neither of the restless state and the normal state is applicable. The restless state may be represented as a plurality of level values. For example, the restless state may be represented by three levels (a strong restless state, a moderate restless state, and a mild restless state). In this case, the higher the level, the more likely it is that a person to be monitored will display problematic behavior, or the more likely it is that he/she will cause a major problem. The unknown state is a state in which it is not possible to easily determine whether the state of the person to be monitored is the restless state or the normal state. The identification model 143 is generated, for example, by learning the relation between past sensor data and a past restless state or non-restless state. The identification model 143 corresponds to the identification model 40 or 50 shown in FIG. 1.

The past data 141 includes learning data used for machine learning of the identification model 143. In the past data 141, a label indicating whether the patient was in the normal state or the restless state when each sensor data was acquired is assigned to past sensor data used to generate the identification model 143. The past data 141 includes past sensor data of a person to be monitored acquired from the sensor group 120. The past data 141 may include sensor data acquired from a patient other than the person to be monitored.

The attribute information 142 includes attribute information of a group to which a patient from whom the sensor data used to generate the identification model 143 is acquired belongs. The attribute information includes, for example, information about a facility in which the patient is hospitalized, information about an area around the facility, and information about time. The information about the facility includes, for example, information indicating the department which is providing the patient with medical care, i.e., neurosurgery, cardiac surgery, respiratory surgery, medical oncology, psychiatry, hospice care, or the like. The information about the facility may include, for example, information about the type of the facility, such as an acute-care hospital, a rehabilitation hospital, a nursing care facility, a senior care facility, or the like. The information about the area around the facility includes information about the location, the region, hospitals etc. in the area around the facility, temperature, humidity, the average age of local residents, kinds of food eaten and drinks drunk in the region, or the like in the area. The information about time includes, for example, information indicating a season or a month, or information indicating a time period in one day, such as day or night.

The sensor group 120 includes one or a plurality of sensors that acquire biological information (sensor data) of a person to be monitored, such as a patient. The sensor data includes information selected from among a group of items including a heartbeat, respiration, a blood pressure, a body temperature, a level of consciousness, a skin temperature, a skin conductance response, an electrocardiographic waveform, and an electroencephalographic waveform. Attribute information 130 includes attribute information of a group to which the person to be monitored belongs. The sensor group 120 and the attribute information 130 correspond to the sensor group 20 and attribute information 30, respectively, shown in FIG. 1.

The restlessness identification apparatus 110 includes an internal state identification unit 111, a determination unit 112, and a model generation unit 113. The internal state identification unit 111 acquires sensor data of a patient to be monitored from the sensor group 120. The internal state identification unit 111 identifies an internal state (a restless state) of the patient based on the acquired sensor data and the identification model 143 stored in the storage device 140. The internal state identification unit 111 may identify the restless state by extracting feature values from the acquired sensor data and applying the extracted feature values to the identification model 143. The internal state identification unit 111 outputs, for example, a score (a restlessness score) indicating the level of the restless state as a result of the identification of the restless state. The internal state identification unit 111 corresponds to the internal state identification means 13 shown in FIG. 1.

The notification unit 150 outputs the result of the identification of the restless state identified by the internal state identification unit 111 to a medical worker or the like. The notification unit 150 may, for example, notify the medical worker or the like that the patient is in the restless state when the restlessness score output by the internal state identification unit 111 is equal to or greater than a predetermined value. The notification unit 150 includes, for example, at least one of a lamp, an image display device, and a speaker, and may use at least one of light, image information, and sound to notify the medical worker or the like that the patient is in the restless state. Specifically, the notification unit 150 may display information indicating that the patient is in the restless state on a display screen of a portable information terminal apparatus such as a smartphone or a tablet carried by the medical worker or the like. Alternatively, the notification unit 150 may notify the medical worker or the like that the patient is in the restless state by voice, for example, through an earphone(s) worn by the medical worker or the like. Further, the notification unit 150 may display information indicating that the patient is in the restless state on a monitor installed at a nurse station or the like, or may notify the medical worker or the like that the patient is in the restless state by using a speaker installed at the nurse station or the like. The notification unit 150 notifies, when the patient is in the restless state before he/she displays problematic behavior, the medical worker about the restless state, whereby the medical worker or the like can provide the patient with care before the patient displays problematic behavior.

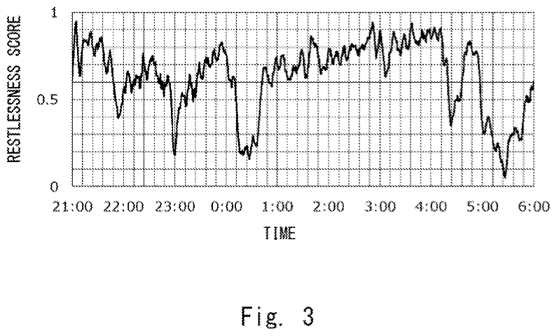

FIG. 3 shows a specific example of the restlessness score. In the graph shown in FIG. 3, the vertical axis indicates the restlessness score and the horizontal axis indicates time. The graph in FIG. 3 shows the changes in the restlessness score with the elapse of time, the restlessness score being obtained as a result of the application of the identification model to sensor data at a certain time in the internal state identification unit 111. In this example, the restlessness score has a value from 0 to 1. It is assumed that the restlessness score “0” indicates that a patient is not in the restless state (the normal state) or that it is least likely that the patient is in the restless state. It is also assumed that the restlessness score “1” indicates that the degree of the restless state is the strongest or that it is most likely that the patient is in the restless state. The above-described identification model is generated, for example, by performing learning using learning data in which a label assigned when the patient is in the normal state is set to “0” and a label assigned when the patient is in the restless state is set to “1”.

The internal state identification unit 111 applies sensor data that can change moment to moment to the identification model 143, and outputs the restlessness score in a time series. When the restlessness score is equal to or greater than a predetermined value, for example, 0.7, the notification unit 150 notifies the medical worker or the like that the patient is in the restless state. A threshold value serving as a criterion for determining the notification may be set as appropriate in accordance with the identification model to be used, other conditions, and the like. The medical worker who receives the notification can go to check on the condition of the patient. The medical worker may input information indicating whether the patient is actually in the restless state or the patient is not actually in the restless state, for example, by using a terminal apparatus such as a tablet placed beside a bed. Further, the medical worker may input information about the details of the treatment applied to the patient, such as an encouraging talk or an adjustment of the bed, by using the terminal apparatus such as the tablet placed beside the bed.

Referring back to FIG. 2, the determination unit 112 determines whether or not a condition for generating another identification model different from an existing identification model is satisfied. The condition for generating another identification model can be replaced with, for example, a condition for regenerating the identification model 143. For example, the determination unit 112 determines whether or not the condition for generating another identification model is satisfied based on the accuracy of the result of the identification of the restless state identified by the internal state identification unit 111. The determination unit 112 determines that the condition for generating another identification model is satisfied, for example, when the accuracy of the result of the identification is lower than a predetermined threshold value. The accuracy of the result of the identification of the restless state can be calculated by comparing the result of the identification performed by the internal state identification unit 111 with the result of the determination of the restless state input by the medical worker or the like.

Alternatively, the determination unit 112 may determine whether or not the condition for generating another identification model is satisfied based on the time series data (the restlessness scores) of the restless state identified by the internal state identification unit 111. The determination unit 112 determines that the condition for generating another identification model is satisfied, for example, when the restlessness scores are distributed within a certain range. Here, the fact that certain restlessness scores are distributed within a certain range means, for example, a state in which the ratio of the number of restlessness scores having values within a certain range to the total number of restlessness scores (all samples) is equal to or greater than a predetermined ratio. For example, when the values of the restlessness scores are concentrated in a particular range, the identification model used to generate the restlessness score may not be able to properly identify the restless state. Specifically, when most of the restlessness scores fall within a range close to an intermediate range between the value indicating a restless state and the value indicating a normal state, the identification model may not be able to correctly identify the restless state and the normal state. The determination unit 112 may determine that the condition for generating another identification model is satisfied when the ratio of the restlessness scores having values of the range close to the intermediate range is equal to or greater than a predetermined ratio.

Further, the determination unit 112 may determine whether or not the condition for generating another identification model is satisfied based on the attribute information 130 of the person to be monitored and the attribute information 142 stored in the storage device 140. The determination unit 112, for example, compares the attribute information 130 with the attribute information 142. The determination unit 112 may determine that the condition for generating another identification model is satisfied when the current situation of the facility differs from that at the time of the generation of the identification model (when the attribute information has changed). For example, when a new hospital is established in the area around the hospital where the patient has been hospitalized or when there are no longer hospitals in the area around the hospital where the patient has been hospitalized, the determination unit 112 may determine that the condition for generating another identification model is satisfied. Alternatively, when a new medical department is added to the hospital where the patient has been hospitalized etc., the determination unit 112 may determine that the condition for generating another identification model is satisfied.

Whether or not the group of the person to be monitored (the attribute information thereof) is different from the group at the time of the generation of the identification model (the attribute information thereof), in other words, whether or not the group has changed, can be determined using, for example, the following method. First, in a learning phase, by using a learning apparatus (not shown), machine learning is performed using the attribute information 130 and the attribute information 142 as explanatory variables and a value (changed: 1, no change: 0) indicating whether or not the group has changed as an objective variable. Training data used for the machine learning can be generated based on the accuracy of the result of the identification performed using the identification model 143 and the sensor data acquired from the sensor group 120. The accuracy of the result of the identification can be calculated, for example, by comparing the value input by the medical worker with the result of the identification. It is assumed that, when the accuracy is lower than a preset threshold value, for example, 70%, the group has changed (a value “1”), while when the accuracy is higher than the threshold value, the group has not changed (a value “0”). In an identification phase, the attribute information 130 and the attribute information 142 are applied to the model obtained by the machine learning, whereby it is possible to obtain a value indicating whether or not the group has changed. In the above case, in the learning phase, the accuracy is calculated to determine whether or not the group has changed. However, in the identification phase, it is possible to determine whether or not the group has changed from the attribute information 130 and the attribute information 142 without calculating the accuracy.

Further, the determination unit 112 specifies the season from the current date and time. The determination unit 112 may determine that the condition for generating another identification model is satisfied when the season has changed. Alternatively, the determination unit 112 may determine that the condition for generating another identification model is satisfied once a month. The determination unit 112 may determine that the condition for generating another identification model is satisfied at a timing when the operation of the biological information processing system 100 is started. The determination unit 112 corresponds to the determination means 11 shown in FIG. 1.

When the determination unit 112 determines that the condition for generating another identification model is satisfied, the model generation unit 113 generates a new identification model separately from the existing identification model 143 by using past sensor data of the person to be monitored acquired from the sensor group 120. The new generated identification model is used in the internal state identification unit 111 to identify the restless state. When it is not determined that the condition for generating another identification model is satisfied, the model generation unit 113 generates no new identification model. When it is not determined that the condition for generating another identification model is satisfied, the data acquired from the sensor group 120 may be added to the past data 141 used to generate the existing identification model 143. The model generation unit 113 corresponds to the model generation means 12 shown in FIG. 1.

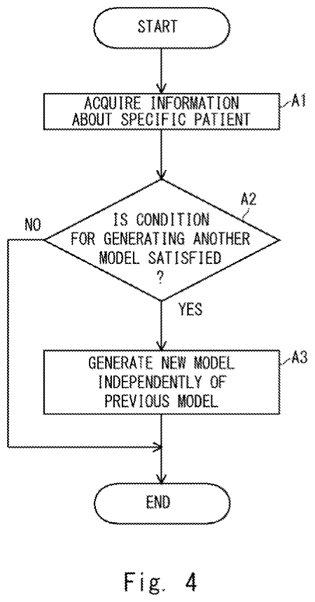

Next, an operation procedure (a biological information processing method) will be described. FIG. 4 shows the operation procedure. The internal state identification unit 111 acquires sensor data from the sensor group 120 (Step A1). The determination unit 112 determines whether or not a condition for generating another identification model is satisfied (Step A2). When it is determined in Step A2 that the condition for generating another identification model is satisfied, the model generation unit 113 generates a new identification model independently of the previous model by using the sensor data of a person to be monitored (Step A3). When it is determined in Step A2 that the condition for generating another identification model is not satisfied, the model generation unit 113 generates no new identification model.

In this example embodiment, when a condition for generating another identification model is satisfied, a new identification model is generated independently of the previous model. For example, when the accuracy of a result of the identification of the restless state is lower than a predetermined threshold value, it is considered that the identification model 143 currently in use may not be suitable for identifying the restless state of the person to be monitored. In such a case, it is determined that the condition for generating another identification model is satisfied, a new identification model is generated independently of the previous model, and then the identification of the restless state is performed by using the generated identification model. By doing so, it is possible to prevent or reduce a decrease in accuracy of the result of the identification of the restless state. Further, when the restlessness scores, which are the results of the identification of the restless state, are concentrated, for example, in a certain range near the center thereof, the identification model currently in use may not be able to correctly identify the restless state of the person to be monitored. In such a case, it is determined that the condition for generating another identification model is satisfied, a new identification model is generated independently of the previous model, and then the identification of the restless state is performed by using the generated identification model, whereby it is possible to prevent or reduce a decrease in accuracy of the result of the identification of the restless state.

When the situation of the facility where the person to be monitored has been hospitalized changes, the attribute information of the group to which the person to be monitored belongs changes, and thus the identification model 143 currently in use may not be suitable for identifying the restless state of the person to be monitored. Further, since the sensor data is affected by the temperature and the humidity of an external environment, the identification model 143 may no longer be suitable for identifying the restless state of the person to be monitored depending on the season and the time. In such a case, it is determined that the condition for generating another identification model is satisfied, a new identification model is generated independently of the previous model, and then the identification of the restless state is performed by using the generated identification model, whereby it is possible to prevent or reduce a decrease in accuracy of the result of the identification of the restless state. In particular, when another identification model is generated in accordance with a change in season or time, it is possible to periodically identify the restless state using an identification model adapted to the season or the time.

For example, assume a case where the identification model 143 is generated by using sensor data acquired from a patient in a certain hospital as learning data. When this identification model 143 is applied to sensor data acquired from a patient hospitalized in another hospital and it is found that the attribute information of the group to which the former patient from whom the sensor data is acquired belongs is similar to the attribute information of the group to which the latter patient from whom the sensor data is acquired belongs, it is considered that the accuracy of the result of the identification of the restless state using the identification model 143 is high. However, for example, when the regions, the times, or the medical departments are different from each other, the accuracy of the result of the identification of the restless state using the identification model 143 is not always high. In this example embodiment, the determination unit 112 determines that the condition for generating another identification model is satisfied, for example, when the region, the time, or the medical department at the time of the generation of the identification model is different from that at the time of the application of the identification model. By separately generating a new identification model and identifying the restless state by using the identification model applied to the person to be monitored, it is possible, in the biological information processing system 100, to prevent or reduce a decrease in accuracy of the result of the identification of the restless state.

Next, a second example embodiment according to the present disclosure will be described. The configuration of a biological information processing system according to this example embodiment may be similar to that of the biological information processing system 100 according to the first example embodiment shown in FIG. 2. In this example embodiment, the determination unit 112 determines whether or not the amount of past sensor data of a person to be monitored included in the past data 141 is equal to or greater than a threshold value. When the determination unit 112 determines that the amount of past sensor data of the person to be monitored is equal to or greater than the threshold value, it determines that a sufficient amount of sensor data of the person to be monitored is present. When the determination unit 112 determines that a sufficient amount of sensor data of the person to be monitored is present, it determines whether or not a condition for generating another identification model is satisfied. When the determination unit 112 determines that an insufficient amount of sensor data of the person to be monitored is present, the determination unit 112 adds the acquired sensor data to the past data 141. Further, the determination unit 112 causes the model generation unit 113 to regenerate the identification model. The configurations other than the above ones may be similar to those of the first example embodiment. The threshold value of the amount of sensor data can be determined based on, for example, whether or not there is almost no change between the accuracy of the identification before the determination is performed by increasing the data and the accuracy of the identification after the determination is performed by increasing the data.

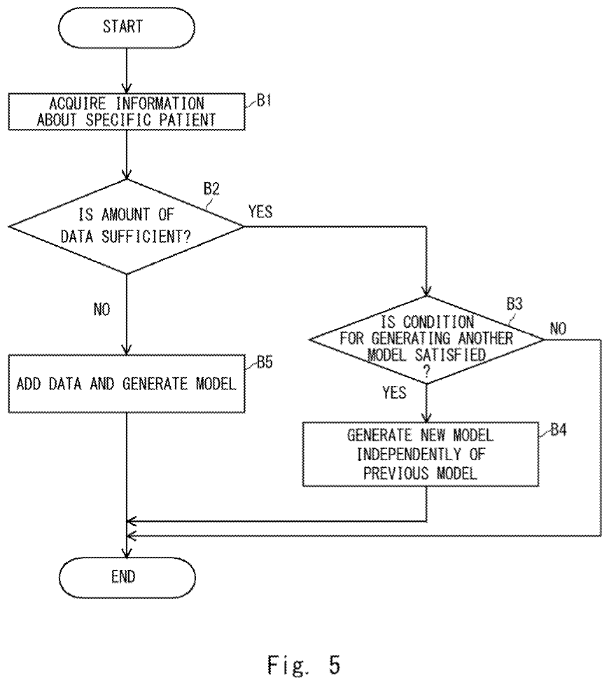

FIG. 5 shows an operation procedure in the second example embodiment. In this example embodiment, the internal state identification unit 111 acquires sensor data from the sensor group 120 (Step B1). Step B1 may be similar to Step A1 shown in FIG. 4. The determination unit 112 determines whether or not the amount of past sensor data of a person to be monitored included in the past data 141 is sufficient to generate the identification model (Step B2). If the determination unit 112 determines in Step B2 that an insufficient amount of data is present, the determination unit 112 adds the sensor data acquired in Step B1 to the past data 141 and causes the model generation unit 113 to regenerate the identification model (Step B5). The model generation unit 113 regenerates (modifies) the identification model 143 used in the internal state identification unit 111 by using the past data 141 to which the sensor data has been added.

If the determination unit 112 determines in Step B2 that a sufficient amount of data is present, it determines whether or not a condition for generating another identification model is satisfied (Step B3). Step B3 may be similar to Step A2 shown in FIG. 4. When it is determined in Step B3 that the condition for generating another identification model is satisfied, the model generation unit 113 generates a new identification model independently of the previous model by using the past sensor data of a person to be monitored (Step B4). Step B4 may be similar to Step A3 shown in FIG. 4. When it is determined in Step B3 that the condition for generating another identification model is not satisfied, the model generation unit 113 generates no new identification model.

In this example embodiment, the determination unit 112 determines whether or not an amount of sensor data sufficient to generate the identification model is present. When the determination unit 112 determines that an amount of sensor data sufficient to generate the identification model is present, it determines whether or not a condition for generating another identification model is satisfied. In a case in which an insufficient amount of sensor data is present, when the identification model is generated, it is considered that the accuracy of the result of the identification of the restless state using the generated identification model is not high. When the determination unit 112 determines that an insufficient amount of sensor data is present, the model generation unit 113 generates no new identification model independently of the previous identification model. In this way, it is possible to prevent or reduce generation of an identification model of which the accuracy of the result of the identification is low and an identification of a restless state using this identification model.

Note that, in the above example embodiments, the storage device 140 can store a plurality of identification models 143 including the identification model generated in Step A3 (see FIG. 4) or Step B4 (see FIG. 5). In this case, the storage device 140 may store, for each identification model, the attribute information 142 of the group from which the sensor data used to generate the identification model is acquired belongs. In a case in which the plurality of identification models 143 that can be used by the internal state identification unit 111 are stored in the storage device 140, the determination unit 112 may calculate the accuracy of the result of the identification of each of the plurality of identification models when each of these models is applied to the acquired sensor data. The determination unit 112 may select an identification model to be used in the internal state identification unit 111 based on the accuracy of the result of the identification. For example, the determination unit 112 may select, from among the plurality of identification models, the identification model with the highest accuracy of the identification result as the identification model to be used to identify a restless state.

In the above example embodiments, the functions of the respective components in the biological information processing system 100 may be implemented by using hardware or software. Further, the functions of the respective components in the biological information processing system 100 may be implemented by combining hardware with software.

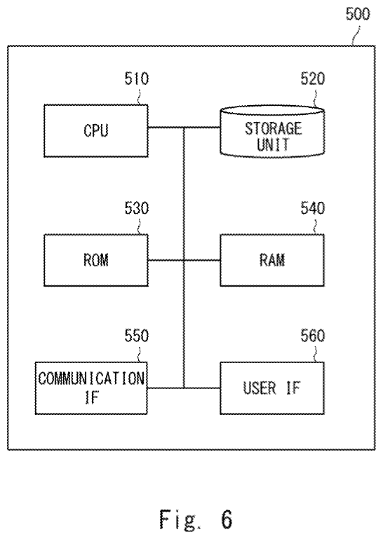

FIG. 6 shows a configuration example of an information processing apparatus (a computer apparatus) that can be used for the restlessness identification apparatus 110. An information processing apparatus 500 includes a control unit (Central Processing Unit (CPU)) 510, a storage unit 520, a Read Only Memory (ROM) 530, a Random Access Memory (RAM) 540, a communication Interface (IF) 550, and a user interface 560.

The communication interface 550 is an interface for connecting the information processing apparatus 500 and a communication network through wired communication means or wireless communication means. The user interface 560 includes a display unit such as a display. Further, the user interface 560 includes input units such as a keyboard, a mouse, and a touch panel.

The storage unit 520 is an auxiliary storage device capable of holding various types of data. The storage unit 520 is not necessarily a part of the information processing apparatus 500, and it may instead be an external storage device or a cloud storage connected to the information processing apparatus 500 through a network. The storage unit 520 corresponds to the storage device 140 shown in FIG. 2. The ROM 530 is a nonvolatile storage device. For example, a semiconductor memory device such as a flash memory having a relatively small capacity is used for the ROM 530. A program executed by the CPU 510 can be stored in the storage unit 520 or the ROM 530.

The aforementioned program can be stored using any type of non-transitory computer readable media and provided to the information processing apparatus 500. Non-transitory computer readable media include any type of tangible storage media. Examples of non-transitory computer readable media include magnetic storage media (such as floppy disks, magnetic tapes, hard disks, etc.), optical magnetic storage media (such as magneto-optical disks), optical disc media (such as CD (compact disc), DVD (digital versatile disc), etc.), and semiconductor memories (such as mask ROM, PROM (programmable ROM), EPROM (erasable PROM), flash ROM, RAM, etc.). Further, the program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

The RAM 540 is a volatile storage device. Various types of semiconductor memory devices such as Dynamic Random Access Memory (DRAM) or Static Random Access Memory (SRAM) are used for the RAM 540. The RAM 540 can be used as an internal buffer for temporarily storing data or the like. The CPU 510 develops the program stored in the storage unit 520 or the ROM 530 in the RAM 540 and executes it. The CPU 510 executes the program, whereby the function of each of the internal state identification unit 111, the determination unit 112, and the model generation unit 113 in the restlessness identification apparatus 110 shown in FIG. 2 is implemented. The CPU 510 may include an internal buffer capable of temporarily storing data or the like.

The example embodiments according to the present disclosure have been described above in detail. However, the present disclosure is not limited to the example embodiments described above, and the example embodiments to which modifications and corrections have been made without departing from the spirit of the disclosure are included in the present disclosure.

For example, the whole or part of the example embodiments disclosed above can be described as, but not limited to, the following supplementary notes.

### Supplementary Note 1

A biological information processing apparatus comprising:

an internal state identification unit configured to acquire sensor data of a person to be monitored from a sensor group including one or a plurality of sensors and identify an internal state of the person to be monitored based on the acquired sensor data and an identification model for identifying the internal state of the person to be monitored, the identification model being generated using sensor data acquired in the past;

a determination unit configured to determine whether or not a condition for generating another identification model different from an existing identification model is satisfied; and

a model generation unit configured to generate, when the determination unit determines that the condition is satisfied, an identification model by using the sensor data of the person to be monitored that is acquired from the sensor group, the identification model being different from the identification model used by the internal state identification unit.

### Supplementary Note 2

The biological information processing apparatus according to Supplementary note 1, wherein the internal state includes whether or not the person to be monitored is in a restless state, and the internal state identification unit outputs levels of the restless state as a result of the identification of the internal state.

### Supplementary Note 3

The biological information processing apparatus according to Supplementary note 1 or 2, wherein the determination unit determines whether or not the condition is satisfied based on accuracy of the result of the identification of the internal state identified by the internal state identification unit.

### Supplementary Note 4

The biological information processing apparatus according to Supplementary note 3, wherein when the accuracy of the result of the identification is lower than a threshold value, the determination unit determines that the condition is satisfied.

### Supplementary Note 5

The biological information processing apparatus according to Supplementary note 2, wherein the determination unit determines whether or not the condition is satisfied based on the levels of the restless state identified by the internal state identification unit.

### Supplementary Note 6

The biological information processing apparatus according to Supplementary note 5, wherein the determination unit determines that the condition is satisfied when the levels of the restless state are distributed within a predetermined range.

### Supplementary Note 7

The biological information processing apparatus according to Supplementary note 1 or 2, wherein the determination unit determines whether or not the condition is satisfied based on attribute information of a group to which the person to be monitored belongs and attribute information of a group to which a person from whom the sensor data acquired in the past is acquired belongs.

### Supplementary Note 8

The biological information processing apparatus according to Supplementary note 7, wherein the attribute information includes information about a facility where a patient is hospitalized, information about an area around the facility where the patient is hospitalized, and information about time.

### Supplementary Note 9

The biological information processing apparatus according to Supplementary note 7 or 8, wherein when the attribute information of the group to which the person to be monitored belongs differs from the attribute information of the group to which the person from whom the sensor data acquired in the past is acquired belongs, the determination unit determines that the condition is satisfied.

### Supplementary Note 10

The biological information processing apparatus according to Supplementary note 9, wherein the determination unit determines whether or not the attribute information of the group to which the person to be monitored belongs differs from the attribute information of the group to which the person from whom the sensor data acquired in the past is acquired belongs by using a model that is generated by performing machine learning using pieces of these attribute information as explanatory variables and information indicating whether or not the pieces of these attribute information differ from each other as an object variable.

### Supplementary Note 11

The biological information processing apparatus according to any one of Supplementary notes 1 to 10, wherein when there are a plurality of identification models that the internal state identification unit is able to use, the determination unit selects the identification model used by the internal state identification unit based on accuracy of the result of the identification of the internal state identified by the internal state identification unit using each of the plurality of identification models.

### Supplementary Note 12

The biological information processing apparatus according to any one of Supplementary notes 1 to 11, wherein the determination unit determines whether or not an amount of the sensor data of the person to be monitored that is acquired from the sensor group is equal to or greater than a threshold value, and when the determination unit determines that the amount of the sensor data is equal to or greater than the threshold value, the determination unit determines whether or not the condition is satisfied.

### Supplementary Note 13

A biological information processing method comprising:

acquiring sensor data of a person to be monitored from a sensor group including one or a plurality of sensors and identifying an internal state of the person to be monitored based on the acquired sensor data and an identification model for identifying the internal state of the person to be monitored, the identification model being generated using sensor data acquired in the past;

determining whether or not a condition for generating another identification model different from an existing identification model is satisfied; and

generating, when it is determined that the condition is satisfied, an identification model by using the sensor data of the person to be monitored that is acquired from the sensor group, the identification model being different from the identification model used to identify the internal state.

### Supplementary Note 14

A computer readable recording medium storing a program for causing a computer to:

acquire sensor data of a person to be monitored from a sensor group including one or a plurality of sensors and identify an internal state of the person to be monitored based on the acquired sensor data and an identification model for identifying the internal state of the person to be monitored, the identification model being generated using sensor data acquired in the past;

determine whether or not a condition for generating another identification model different from an existing identification model is satisfied; and

generate, when it is determined that the condition is satisfied, an identification model by using the sensor data of the person to be monitored that is acquired from the sensor group, the identification model being different from the identification model used to identify the internal state.

### REFERENCE SIGNS LIST

### 10 BIOLOGICAL INFORMATION PROCESSING APPARATUS

### 11 DETERMINATION MEANS

### 12 MODEL GENERATION MEANS

### 13 INTERNAL STATE IDENTIFICATION MEANS

### 20 SENSOR GROUP

### 30 ATTRIBUTE INFORMATION

### 40, 50 IDENTIFICATION MODEL

### 100 BIOLOGICAL INFORMATION PROCESSING SYSTEM

### 110 RESTLESSNESS IDENTIFICATION APPARATUS

### 111 INTERNAL STATE IDENTIFICATION UNIT

### 112 DETERMINATION UNIT

### 113 MODEL GENERATION UNIT

### 120 SENSOR GROUP

### 130 ATTRIBUTE INFORMATION

### 140 STORAGE DEVICE

### 141 PAST DATA

### 142 ATTRIBUTE INFORMATION

### 143 IDENTIFICATION MODEL

### 150 NOTIFICATION UNIT

What is claimed is:

1. A biological information processing apparatus comprising:
a memory storing instructions, and
a processor configured to execute the instructions to implement;
an internal state identification unit configured to acquire sensor data of a person to be monitored from a sensor group including one or a plurality of sensors and identify an internal state of the person to be monitored based on the acquired sensor data and an identification model for identifying the internal state of the person to be monitored, the identification model being generated using sensor data acquired in the past;
a determination unit configured to determine whether or not a condition for generating another identification model different from an existing identification model is satisfied; and
a model generation unit configured to generate, when the determination unit determines that the condition is satisfied, an identification model by using the sensor data of the person to be monitored that is acquired from the sensor group, the identification model being different from the identification model used by the internal state identification unit.

2. The biological information processing apparatus according to claim 1, wherein the internal state includes whether or not the person to be monitored is in a restless state, and the internal state identification unit outputs levels of the restless state as a result of the identification of the internal state.

3. The biological information processing apparatus according to claim 1, wherein the determination unit determines whether or not the condition is satisfied based on accuracy of the result of the identification of the internal state identified by the internal state identification unit.

4. The biological information processing apparatus according to claim 3, wherein when the accuracy of the result of the identification is lower than a threshold value, the determination unit determines that the condition is satisfied.

5. The biological information processing apparatus according to claim 2, wherein the determination unit determines whether or not the condition is satisfied based on the levels of the restless state identified by the internal state identification unit.

6. The biological information processing apparatus according to claim 5, wherein the determination unit determines that the condition is satisfied when the levels of the restless state are distributed within a predetermined range.

7. The biological information processing apparatus according to claim 1, wherein the determination unit determines whether or not the condition is satisfied based on attribute information of a group to which the person to be monitored belongs and attribute information of a group to which a person from whom the sensor data acquired in the past is acquired belongs.

8. The biological information processing apparatus according to claim 7, wherein the attribute information includes information about a facility where a patient is hospitalized, information about an area around the facility where the patient is hospitalized, and information about time.

9. The biological information processing apparatus according to claim 7, wherein when the attribute information of the group to which the person to be monitored belongs differs from the attribute information of the group to which the person from whom the sensor data acquired in the past is acquired belongs, the determination unit determines that the condition is satisfied.

10. The biological information processing apparatus according to claim 9, wherein the determination unit determines whether or not the attribute information of the group to which the person to be monitored belongs differs from the attribute information of the group to which the person from whom the sensor data acquired in the past is acquired belongs by using a model that is generated by performing machine learning using pieces of these attribute information as explanatory variables and information indicating whether or not the pieces of these attribute information differ from each other as an object variable.

11. The biological information processing apparatus according to claim 1, wherein when there are a plurality of identification models that the internal state identification unit is able to use, the determination unit selects the identification model used by the internal state identification unit based on accuracy of the result of the identification of the internal state identified by the internal state identification unit using each of the plurality of identification models.

12. The biological information processing apparatus according to claim 1, wherein the determination unit determines whether or not an amount of the sensor data of the person to be monitored that is acquired from the sensor group is equal to or greater than a threshold value, and when the determination unit determines that the amount of the sensor data is equal to or greater than the threshold value, the determination unit determines whether or not the condition is satisfied.

13. A biological information processing method comprising:
acquiring sensor data of a person to be monitored from a sensor group including one or a plurality of sensors and identifying an internal state of the person to be monitored based on the acquired sensor data and an identification model for identifying the internal state of the person to be monitored, the identification model being generated using sensor data acquired in the past;
determining whether or not a condition for generating another identification model different from an existing identification model is satisfied; and
generating, when it is determined that the condition is satisfied, an identification model by using the sensor data of the person to be monitored that is acquired from the sensor group, the identification model being different from the identification model used to identify the internal state.

14. A non-transitory computer readable recording medium storing a program for causing a computer to:
acquire sensor data of a person to be monitored from a sensor group including one or a plurality of sensors and identify an internal state of the person to be monitored based on the acquired sensor data and an identification model for identifying the internal state of the person to be monitored, the identification model being generated using sensor data acquired in the past;
determine whether or not a condition for generating another identification model different from an existing identification model is satisfied; and
generate, when it is determined that the condition is satisfied, an identification model by using the sensor data of the person to be monitored that is acquired from the sensor group, the identification model being different from the identification model used to identify the internal state.
